SEQUENCE LISTING (3 sequences)

SEQ ID NO: 1 - protein, 167 aa, Artificial Sequence
  Amino acid sequence of recombinant interferon
MCDLPQTHSLGNRRALILLAQMRRISPFSCLKDRHDFGFPQEEFDGNQFQKAQAISVLHEMIQQTFNLFSTKDSSAAWDESLLEKFYTELYQQLNDLEACVIQEVGVEETPLMNVDSILAVKKYFQRITLYLTEKKYSPCAWEVVRAEIMRSFSLSTNLQERLRRKE

SEQ ID NO: 2 - DNA, 504 bp, Artificial Sequence
  Nucleotide sequence encoding recombinant interferon
atgtgcgacctgccgcagacccactccctgggtaaccgtcgtgctctgatcctgctggctcagatgcgtcgtatctccccgttctcctgcctgaaagaccgtcacgacttcggtttcccgcaggaagaattcgacggtaaccagttccagaaagctcaggctatctccgttctgcacgaaatgatccagcagaccttcaacctgttctccaccaaagactcctccgctgcttgggacgaatccctgctggaaaaattctacaccgaactgtaccagcagctgaacgacctggaagcttgcgttatccaggaagttggtgttgaagaaaccccgctgatgaacgttgactccatcctggctgttaaaaaatacttccagcgtatcaccctgtacctgaccgaaaaaaaatactccccgtgcgcttgggaagttgttcgtgctgaaatcatgcgttccttctccctgtccaccaacctgcaggaacgtctgcgtcgtaaagaataa

SEQ ID NO: 3 - DNA, 504 bp, Artificial Sequence
  Nucleotide sequence encoding recombinant interferon
tacacgctggacggcgtctgggtgagggacccattggcagcacgagactaggacgaccgagtctacgcagcatagaggggcaagaggacggactttctggcagtgctgaagccaaagggcgtccttcttaagctgccattggtcaaggtctttcgagtccgatagaggcaagacgtgctttactaggtcgtctggaagttggacaagaggtggtttctgaggaggcgacgaaccctgcttagggacgacctttttaagatgtggcttgacatggtcgtcgacttgctggaccttcgaacgcaataggtccttcaaccacaacttctttggggcgactacttgcaactgaggtaggaccgacaattttttatgaaggtcgcatagtgggacatggactggcttttttttatgaggggcacgcgaacccttcaacaagcacgactttagtacgcaaggaagagggacaggtggttggacgtccttgcagacgcagcatttcttatt

(19) United States
(12) Patent Application Publication
(10) Pub. No.: US 2013/0273527 A1
(43) Pub. Date: Oct. 17, 2013

(54) METHOD FOR INDUCING CELL SENESCENCE BY RECOMBINANT INTERFERON WITH ALTERED SPATIAL CONFIGURATION

(71) Applicant: SUPERLAB FAR EAST LIMITED, TORTOLA (VG)

(72) Inventors: Guangwen Wei, Sichuan (CN); Huiling Li, Chongqing (CN); Dongqin Yang, Shanghai (CN); Xin Cao, Shanghai (CN); Hongling Huang, Shanghai (CN); Miao Ding, Shanghai (CN); Haineng Xu, Shanghai (CN); Xinyuan Liu, Shanghai (CN); Mingyang Li, Chongqing (CN)

(73) Assignee: SUPERLAB FAR EAST LIMITED, TORTOLA (VG)

(21) Appl. No.: 13/861,617

(22) Filed: Apr. 12, 2013

Related U.S. Application Data
(60) Provisional application No. 61/623,764, filed on Apr. 13, 2012.

(51) Int. Cl. C12N 5/09; G01N 33/574; G01N 33/50

(52) U.S. Cl. CPC C12N 5/0693; G01N 33/5011; G01N 33/57496
USPC 435/6.1; 435/375; 435/29; 435/7.1; 435/18; 435/7.4

(57) ABSTRACT

This invention provides a method for inducing cell senescence by recombinant interferon with altered spatial configuration, wherein the interferon is encoded by a nucleotide sequence having the sequence of SEQ ID NO.2.

### FIELD OF THE INVENTION

This invention relates in general to a method for inducing cell senescence by recombinant interferon with altered spatial configuration.

### BACKGROUND OF THE INVENTION

Cancer is a leading cause of death worldwide and the total number of cases globally is increasing. According to statistics of WHO, the number of global cancer deaths is projected to increase 45% from 2007 to 2030 (from 7.9 million to 11.5 million deaths). New cases of cancer in the same period are estimated to jump from 11.3 million in 2007 to 15.5 million in 2030. Of which, the morbidity and mortality of lung cancer take the first place in the worldwide malignant tumors, and there is a growing trend of the lung cancer with the deterioration of the environment.

Cell senescence means that the cells enter irreversibly into a relatively stable state after occurrence of cell cycle arrest and loss of proliferation capacity. There are many factors inducing cell senescence, such as: activation of oncogene, telomere shortening, oxygen pressure and genetic damage. In the pathological change of cancer, cell senescence plays a key role, for example, activation of oncogene arising from gene mutation will stimulate most of normal cells to start their senescence path; as a precondition that normal cell is turned into tumor cell, the cell senescence system must be bypassed, namely, cell senescence is an very important mechanism for inhibiting cancer in the body and also a major carrier of resisting cancer in the body. The lastest researches show that, inducing cell senescence has become a new tactic for curing cancer, other than inhibiting the cancer.

Interferon (IFN) is a kind of soluble protein produced by a variety of cells, which has many important biological functions, including anti-viral, anti-tumor, and immunoregulatory functions. Interferons can be divided into type I, type II, and type III interferons according to the types of producing cells, receptors and biological activities etc. Type I IFNs, which are mostly induced by viruses and synthetic double-stranded RNA, are also known as anti-viral interferons. There are three forms of type I interferons: IFNα, INFβ, IFNω. Type II IFN, also known as immune interferon or IFNγ, is produced by T cells, and is an important immunoregulatory factor in vivo. Type III interferon is made up of IFN-λ molecules.

In recent years, companies throughout the world have engaged in interferon research, as exemplified by a number of pertinent patents and disclosure documents. For example, U.S. Pat. Nos. 4,695,623 and 4,897,471 disclosed new types of human interferon polypeptides, the amino acid sequence of which contains the common or predominant amino acids found in naturally occurring α-interferon polypeptides. That new type of interferon was named IFN-con (consensus interferon α). The disclosed amino acid sequences were named IFN-con1, IFN-con2 and IFN-con3. Genes encoding consensus interferon sequences, i.e. ‘IFN-cons,’ as well as means of gene expression in Escherichia coli were also disclosed. Compared with leukocyte interferon or other type I interferons, studies have shown that recombinant IFN-con has higher anti-viral, anti-proliferative and natural killer cell activity in vitro.

U.S. Pat. No. 5,372,808 disclosed using human IFN-con in the treatment of disease. Compared with previous clinically approved a-interferon such as INTRONA® (IFN-α2b, SGP), recombinant human IFN-con has been shown to have lower side-effects. By the end of 1997, the FDA had approved the use of human IFN-con, which was produced by Amgen and sold under the brand name INFERGEN® (interferon alfacon-1), for clinical treatment of hepatitis C.

U.S. Pat. Nos. 7,364,724 and 7,585,647 disclosed a novel recombinant interferon (hereafter referred to as “rSIFN-co” or “sIFNα”) that has enhanced efficacy, fewer side-effects and can be used in high doses. The recombinant interferon disclosed in the '724 patent has the same amino acid sequence as INFERGEN®, but has different spatial structure and biological efficacy. It is of interest to determine if the rSIFN-co could induce cell senescence and also cure cancer by means of inducing cell senescence.

### SUMMARY OF THE INVENTION

This invention provides a method to induce cell senescence by a recombinant interferon, comprising the step of contacting target cells with the recombinant interferon, wherein the interferon is encoded by a nucleotide sequence having the sequence of SEQ ID NO.2.

### BRIEF DESCRIPTION OF THE DRAWINGS

FIG. 1 shows the cytopathic effect of sIFNα or IFNα2b on A549 cell measured by crystal violet staining.

FIG. 2 shows the cell vibility change of A549 cell analysed by MTT assay. The results were displayed as mean±SD values from four replicate wells of three separated experiments.

FIG. 3 shows A549 cell morphology at day 3 after sIFNα or IFNα2b treatment (Scale bar=50 μm).

FIG. 4 shows A549 cell nuclear morphology observed by Hoechst 33258 staining (Scale bar=10 μm).

FIG. 5 shows western blot analysis of apoptosis-related proteins of A549 cell at day 3 after sIFNα or IFNα2b treatment.

FIG. 6 shows result of Tunel staining on A549 cell at day 3 after sIFNα or IFNα2b treatment (Scale bar=100 μm).

FIG. 7 shows result of Senescence-associated β-galactosidase (SA-β-gal) staining on A549 cell at day 3 after sIFNα or IFNα2b treatment (Scale bar=20 μm).

FIG. 8 shows result of sIFNα inhibit tumor growth of A549 xenograft. (A) Different tumor volumes between different treatments. Points represents mean values (n=8); bars represents SD. (B) At the end of animal experiment, tumors from the mice were dissected and weighted, the average tumor weight was calculated and expressed as mean±SD (n=8, **p<0.01, ***p<0.001).

FIG. 9 shows result of immunostaining of p53 and p21 on A549 cell after sIFNα or IFNα2b treatment (Scale bar=100 μm).

### DETAILED DESCRIPTION OF THE INVENTION

This invention provides a method to induce cell senescence by a recombinant interferon, comprising the step of contacting target cells with the recombinant interferon, wherein the interferon is encoded by a nucleotide sequence having the sequence of SEQ ID NO.2. In one embodiment, the cells are cancer cells. In one embodiment, the interferon does not induce cell apoptosis. In one embodiment, the interferon exhibits anti-proliferation effect in lung cancer cell A549. In another embodiment, the method further comprises the step of examining the cells by crystal violet staining. In another embodiment, the method further comprises the step of examining the cells by Hoeschst 33258 staining. In one embodiment, the cells possess enlarged cell size and flatted shape in morphology. In one embodiment, the cells do not exhibit any nucleus morphologic changes. In one embodiment, the number of cells are reduced. In another embodiment, the method further comprises the step of examining the cells for caspase 3 or caspase 8 expression. In one embodiment, the cells do not exhibit any change in caspase 3 or caspase 8 expressions. In another embodiment, the method further comprises the step of examining the cells by p53 or p21 immunostaining. In one embodiment, the cells exhibit increased expression of p53 or p21. In another embodiment, the method further comprises the step of examining the cells by Tunel staining. In another embodiment, the method further comprises the step of examining the cells by SA-β-gal staining assay. In one embodiment, the cells become SA-β-gal positive.

In one embodiment, the amino acid sequence of the present recombinant interferon, as well as the nucleotide sequence encoding the same, are shown below:

| (SEQ ID NO: 1) | |
|---|---|
| | M C D L P Q T H S L G N R R A L I L L A |
| 1 | ATGTGCGACC TGCCGCAGAC CCACTCCCTG GGTAACCGTC GTGCTCTGAT CCTGCTGGCT |
| | TACACGCTGG ACGGCGTCTG GGTGAGGGAC CCATTGGCAG CACGAGACTA GGACGACCGA |
| | Q M R R I S P F S C L K D R H D F G F P |
| 61 | CAGATGCGTC GTATCTCCCC GTTCTCCTGC CTGAAAGACC GTCACGACTT CGGTTTCCCG |
| | GTCTACGCAG CATAGAGGGG CAAGAGGACG GACTTTCTGG CAGTGCTGAA GCCAAAGGGC |
| | Q E E F D G N Q F Q K A Q A I S V L H E |
| 121 | CAGGAAGAAT TCGACGGTAA CCAGTTCCAG AAAGCTCAGG CTATCTCCGT TCTGCACGAA |
| | GTCCTTCTTA AGCTGCCATT GGTCAAGGTC TTTCGAGTCC GATAGAGGCA AGACGTGCTT |
| | M I Q Q T F N L F S T K D S S A A W D E |
| 181 | ATGATCCAGC AGACCTTCAA CCTGTTCTCC ACCAAAGACT CCTCCGCTGC TTGGGACGAA |
| | TACTAGGTCG TCTGGAAGTT GGACAAGAGG TGGTTTCTGA GGAGGCGACG AACCCTGCTT |
| | S L L E K F Y T E L Y Q Q L N D L E A C |
| 241 | TCCCTGCTGG AAAAATTCTA CACCGAACTG TACCAGCAGC TGAACGACCT GGAAGCTTGC |
| | AGGGACGACC TTTTTAAGAT GTGGCTTGAC ATGGTCGTCG ACTTGCTGGA CCTTCGAACG |
| | V I Q E V G V E E T P L M N V D S I L A |
| 301 | GTTATCCAGG AAGTTGGTGT TGAAGAAACC CCGCTGATGA ACGTTGACTC CATCCTGGCT |
| | CAATAGGTCC TTCAACCACA ACTTCTTTGG GGCGACTACT TGCAACTGAG GTAGGACCGA |
| | V K K Y F Q R I T L Y L T E K K Y S P C |
| 361 | GTTAAAAAAT ACTTCCAGCG TATCACCCTG TACCTGACCG AAAAAAAATA CTCCCCGTGC |
| | CAATTTTTTA TGAAGGTCGC ATAGTGGGAC ATGGACTGGC TTTTTTTTAT GAGGGGCACG |
| | A W E V V R A E I M R S F S L S T N L Q |
| 421 | GCTTGGGAAG TTGTTCGTGC TGAAATCATG CGTTCCTTCT CCCTGTCCAC CAACCTGCAG |
| | CGAACCCTTC AACAAGCACG ACTTTAGTAC GCAAGGAAGA GGGACAGGTG GTTGGACGTC |
| | E R L R R K E |
| (SEQ ID NO: 2) | |
| 481 | GAACGTCTGC GTCGTAAAGA ATAA |
| (SEQ ID NO: 3) | |
| | CTTGCAGACG CAGCATTTCT TATT |

The circular dichroism spectrum (CD) of the present recombinant interferon in ranges of 190-250 nm and 250-320 nm is different from the corresponding CD of INFERGEN® when determined under the same conditions. Several subjects whose BMI ranged from 18 to 23 were chosen to receive this recombinant interferon by intramuscular injection, then graphical charts plotting time of blood collection versus concentration of 2-5 A oligonucleotidase (also referred to as 2′,5′-OAS) in the serum of the subjects were made. The charts shows general two-peak pattern, and the resulting peak area of this chart is greater than that of INFERGEN® after injection under the same conditions. The half-life period of this recombinant interferon in the body is longer than that of INFERGEN® after injection into the body.

The experimental results have also confirmed that the present recombinant interferon is more effective than any interferon used clinically at present (including INFERGEN®). For example, the recombined interferon from this invention is not only capable of restraining DNA replication of HBV, but also of inhibiting secretions of both HBsAg and HBeAg. The efficiency of restraining DNA replication of HBcAg in this interferon is as much as twice of INFERGEN®. The cytotoxicity of the present recombinant interferon is only ⅛ that of currently used interferons, but its antiviral activity is as much as 5-20 times greater than said clinical interferons; meanwhile, the present recombinant interferon is more effective, more broad-spectrum and more lasting in biological responses in human body.

Thus, the present recombinant interferon has different spatial configuration, enhanced biologic activities and different pharmacokinetics characteristics as compared with INFERGEN®.

Therefore, in one embodiment, the present recombinant interferon comprises the amino acid sequence of SEQ ID NO: 1 and is encoded by the nucleotide sequence comprising SEQ ID NO: 2. Further, the present recombinant interferon has the amino acid sequence of SEQ ID NO: 1, and is encoded by the nucleotide sequence of SEQ ID NO: 2. In comparison with an interferon which has the same amino acid sequence with SEQ ID NO: 1, but is not encoded by the nucleotide sequence of SEQ ID NO: 2, such as INFERGEN® (interferon alfacon-1), the present recombinant interferon has different spatial configuration and/or enhanced biologic activities and/or different pharmacokinetics characteristics. For example, the present recombinant interferon has different spatial configuration and enhanced biologic activities, different spatial configuration and different pharmacokinetics characteristics, or enhanced biologic activities and different pharmacokinetics characteristics. Further, said different spatial configuration includes: the circular dichroism spectrum (CD) of the present recombinant interferon in ranges of 190-250 nm and/or 250-320 nm is different from the corresponding CD of INFERGEN® (interferon alfacon-1) when determined under the same conditions. The enhanced biologic activities include: this recombinant interferon has enhanced antiviral activity, greatly reduced toxic side effects and/or could be used in large dosages (each dose >10 million IU). The different pharmacokinetics characteristics include: several subjects whose BMI ranged from 18 to 23 are chosen to receive this recombinant interferon by intramuscular injection, then graphical charts plotting time of blood collection versus concentration of 2-5 A oligonucleotidase in the serum of the subjects are made, and the resulting peak area of this chart is greater than that of INFERGEN® (interferon alfacon-1) after injection under the same conditions and/or the half-life period of this recombinant interferon in the body is longer than that of INFERGEN® (interferon alfacon-1) after injection into the body.

In another embodiment, the present recombinant interferon can be produced by the method comprising the following steps: introducing into an isolated host cell a nucleotide sequence comprising SEQ ID NO: 2 that encodes a recombinant interferon; culturing the host cell in an appropriate condition for the expression of the recombinant interferon; and harvesting the recombinant interferon, wherein the recombinant interferon has an amino acid sequence of SEQ ID NO: 1, and the recombinant interferon inhibits secretion of HBsAg and HBeAg of Hepatitis B Virus. Further, said host cell is Escherichia coli, such as Escherichia coli LGM 194. In some embodiments, the nucleotide sequence comprising SEQ ID NO.2 is under the control of promoter P_{BAD}. In some embodiments, the harvesting step comprises extraction of interferon from fermentation broth, collection of inclusion body, denaturation and renaturation of the harvested protein. In some embodiments, the harvesting step comprises separation and purification of the recombinant interferon.

As used herein, the term “cell senescence” means that the cells center into a relatively stable state after occurrence of cell cycle arrest and loss of proliferation capacity. As some sequences of the telomere will be lost from each division of normal cells, approaching to senescence is an inevitable result of normal cells and also one of potential reasons for body senescence. In addition to shortening of telomere, many factors could induce the rapid senescence of normal cells. The senile cells are generally of big and flat shape, particles in the cells will increase, along with growing number of lysosomes and increased expression activity of pH-dependent β-galactosidase. The most common test method for cell senescence is cell senescence β-galactosidase staining, which is also called a golden standard for testing cell senescence.

As used herein, the term “crystal violet” refers to a basic dye that can be combined with DNA in the nucleus for nucleus staining.

As used herein, the term “MTT” refers to 3-(4,5-Dimethylthiazol-2-yl)2,5-diphenyltetrazolium bromide, which is a yellow stain. MTT assay is also called as MTT colorimetry, which is used to test the survival and growth of cells. The assay principle is that, the succinate dehydrogenase in the mitochondria of living cells allows to reduce exogenous MTT into water-insoluble royal purple crystalline Formazan and deposit in the cells, but dead cells don't have such functions. DMSO can dissolve Formazan in the cells; the light absorbance at 570 nm wavelength is measured by ELISA Reader, and the number of living cells could be reflected indirectly. Within the range of a certain number of cells, the amount of MTT crystal is proportional to the number of cells.

As used herein, the term “Hoechst 33258” refers to a DNA-specific dye combined with A-T bond; such dye enables immediate staining of dead cells or those cells fixed by 70% cold ethanol. The staining of living cells is progressive, and saturated within 10 min. Under a fluorescent microscope, the living nucleus show uniform diffusing fluorescence; in the case of cell apoptosis, dense granular fluorescence can be observed in the nucleus or cytoplasm, or the nucleus is lobulated, fragmented with set of edges.

As used herein, TUNEL staining (i.e.: TUNEL apoptosis testing) is used to test DNA fragmentation in the cell apoptotic process using non-isotopic method, helping to test rapidly individual apoptotic cells in-situ. According to the principle, in the case of apoptosis, the endogenous nuclease is activated, so that double chains of DNA are broken or one chain has a nick, generating a series of 3′-OH ends; under the action of terminal deoxynucleotidyl transferase (TdT), in-situ DNA incision of histiocyte could be labeled with biotin-dUTP, i.e.: TUNEL (Terminal deoxynucleotidyl Transferase Biotin-dUTP Nick End Labeling), which can be bonded specifically with the streptavidin coupled to horseradish peroxidase (Streptavidin-HRP); in the presence of HRP substrate diaminobenzidine (DAB), it's shown as brown (peroxidase activity), allowing to specifically locate the cells being apoptotic, then observe and count these cells under common microscope; given that no DNA fragmentation occurs in the cells under proliferation, 3′-OH isn't formed, almost without staining.

The invention being generally described, will be more readily understood by reference to the following examples which are included merely for purposes of illustration of certain aspects and embodiments of the present invention, and are not intended to limit the invention. Modifications may be made in the design and arrangement of the elements described herein without departing from the scope of the invention as expressed in the appended claims.

Throughout this application, various references or publications are cited. Disclosures of these references or publications in their entireties are hereby incorporated by reference into this application in order to more fully describe the state of the art to which this invention pertains. It is to be noted that the transitional term “comprising”, which is synonymous with “including”, “containing” or “characterized by”, is inclusive or open-ended and does not exclude additional, un-recited elements or method steps.

### EXAMPLES

### Experimental Materials

Superior interferon (sIFNα) is provided by Sichuan Huiyang Life Science and Technology Corp.; IFNα2b is provided by Shanghai Huaxin High Biotechnology Inc.; MTT and DMSO are procured from Sigma; cell culture fluid RPMI1640 and FBS are procured from GIBCO-BRL; TRIzol Reagent is produced by Invitrogen; RevertAid™ First Strand cDNA Synthesis Kit is produced by Fermentas; SYBR Green Realtime PCR Master Mix is produced by TOYOBO; TACS TdT Kit In Situ Apoptosis Detection Kit is procured from R&D; rabbit or mouse ABC Staining System is procured from Santa Cruz; Senescence β-Galactosidase Staining Kit is procured from Beyotime Institute of Biotechnology; PVDF membrane is procured from Millipore; and BSA is procured from Shanghai Sibas Biotechnology Development Co., Ltd.

### Cell Strain and Culture

Human lung adenocarcinoma cell line A549 is procured from American Type Culture Collection (ATCC, Rockvill, Md., USA), of which the culture fluid is RPMI-1640 containing 10% FBS, and the culture condition is 37° C., 5% CO₂ constant temperature incubator.

### Antibodies

Anti-p53 is procured from Eptomics; Anti-p21 from CST; Anti-p27 from CST; Anti-pRB from Santa Cruz; Anti-p15 from CST; Anti-ki67 from Eptomics; Anti-caspase-3 from Santa Cruz; Anti-caspase-8 from CST; and Anti-GAPDH from Santa Cruz.

### Experimental Animals

4-week female BALB/c nude mice are procured from Laboratory Animal Center of Shanghai Institutes for Biological Sciences.

### Cell Culture and Medication

Cell recovery: conduct routine disinfection in the cell lab, exposed to ultraviolet rays over 30 min, and ventilate 10 min by starting the super clean bench; put fresh culture medium in 37° C. constant temperature water bath, and then spray 70% alcohol and wipe off, next transfer into a sterile bench; transfer 10 ml fresh culture medium (containing: 10% FBS RPMI-1640) into a sterile cell culture flask; take out freezing tube from liquid nitrogen storage tank, and put immediately into 40° C. water bath, shake slightly to make complete freezing quickly, and wipe the exterior of the freezing tube by 70% alcohol, then transfer into a sterile bench; transfer the cell suspension into a cell culture flask into which culture medium is already added, and next put into CO₂ incubator; after cells are adhered onto wall (generally 4 h, depending on the type of cells), discard the culture medium containing cryoprotectant, and add fresh culture medium for continuous culturing.

Cell passaging: discard old culture fluid; flush the cells twice slightly by PBS buffer, and try to wash away the residual serum and remove PBS; add 0.25% pancreatin (full of the bottle bottom) for digestion at 37° C., and observe it under inverted phase contrast microscope, finding out that the cell process is retracted until it's almost round, and cell gap is increased; add directly RPMI-1640 culture medium (or serum) containing 10% fetal calf serum to stop digestion; beat upon uniformly cells by elbow suction pipe; the action shall be moderate to avoid bubbling; centrifugate 5 min (1500 rpm) to remove supemate; add PBS bugger to beat upon uniformly and then centrifugate once more; subculture by 1:2 with RPMI-1640 culture fluid resuspension cell containing 10% fetal calf serum; supplement culture fluid to 1/10 of the flash volume; place in the incubator for culturing.

Medication: the cells in sound growing state are paved onto cell culturing plate of proper size, with its initial density about 30% of the culturing palte, and put into the incubator for culturing; after the cells are fully adhered onto the wall on the next day, conduct medication; firstly, dissolve sIFNα or IFNα2b into the culture fluid by desired concentration, remove old culture fluid in the cell culturing plate, and add culture fluid after medication, then put into the incubator for continuous culturing. Fresh culture medium is replaced for the control group.

### Statistical Analysis

Statistical software GraphPad PRISM 4.0 is introduced; the experimental data is expressed by “mean value±standard deviation”, the analysis of difference between groups is tested using independent sample t; when P is smaller than or equal to 0.05, the difference is of statistical significance; when P is smaller than or equal to 0.01, the difference is of great statistical significance.

### Example 1

### Crystal Violet Assay

A549 cells are paved on 24-pore plate, and treated separately by 0 μg/ml, 0.1 μg/ml, 1 μg/ml, 7 μg/ml sIFNα or IFNα2b on the next day, and put into the incubator for 3 d culturing; next taken out from the incubator, and the culture fluid is discarded; 200 μl crystal violet staining solution is added into every mesh (2% crystal violet solution is dissolved in 20% methanol solution) for 15 min staining; the staining solution is washed off carefully, then dried up and photographed for storage.

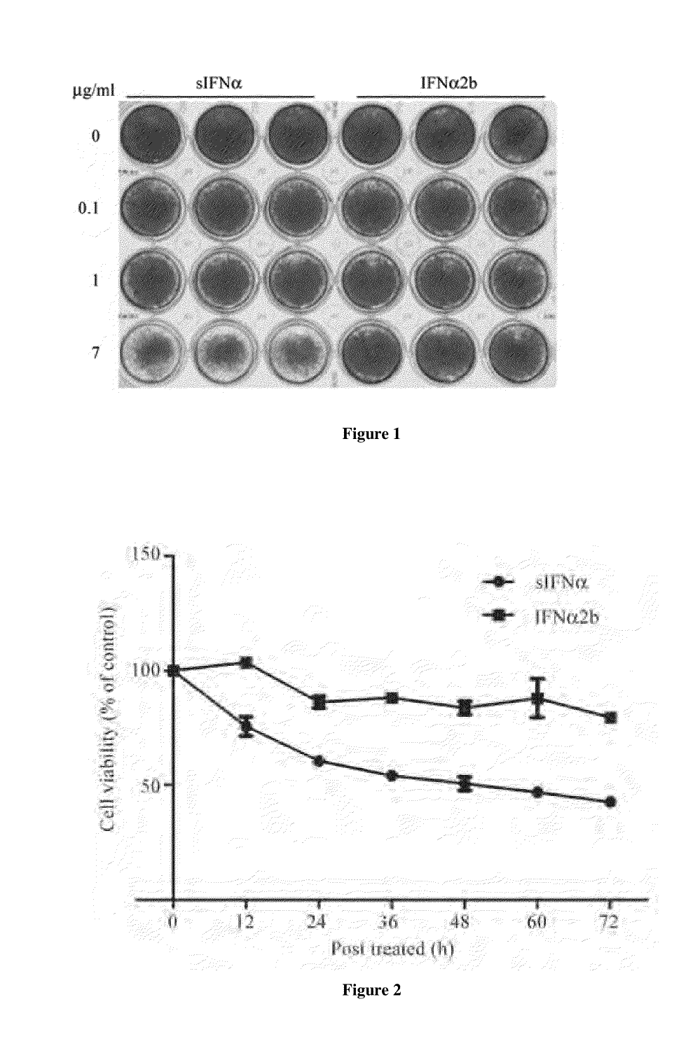

The effect of sIFNα on A549 cell is tested preliminarily using the crystal violet staining method. As compared with the blank control group and the common IFNα2b treatment group clinically used, it's found that the number of cells in sIFNα treatment group is much smaller (FIG. 1).

### Example 2

### Measurement of Cell Viability by MTT Method

Preparation methods of MTT solution: the concentration of MTT solution is generally 5 mg/ml. Firstly, MTT 0.5 g is measured and dissolved into 100 ml PBS buffer, then filtered by 0.22 μm filter membrane to remove the bacteria in the solution, and next placed at 4° C. for storage in dark place. In the process of preparation and storage, the container is preferably wrapped by aluminum foil. Besides, MTT is generally prepared for immediate use, stored in dark place at 4° C. after filtering, and kept valid within 2 weeks, or prepared into 5 mg/ml and stored at −20° C. for a longer period; but repeated freezing and thawing shall be avoided, while it's preferably packaged in small dose, and wrapped in aluminum foil to protect against optical decomposition.

MTT experimental steps: A549 cells are inoculated on 96-pore plate, treated separately by 7 μg/ml sIFNα or IFNα2b on the next day (other than the treatment group); 20 μl MTT solution (5 mg/ml) is added into every mesh at different time (12 h, 24 h, 36 h, 48 h, 60 h and 72 h), then placed at 37° C. for over 3 h culturing; next, the culture fluid containing MTT is removed, and 100 μl DMSO is added into every mesh, placed onto a shaker for 10 min oscillation until the purple substance at bottom is fully dissolved; the light absorbance (D) at 570 nm is measured on ELISA Reader (Thermo MK3, USA). The cell viability=D₅₇₀ (samples)/D₅₇₀(control)×100%.

MTT assay has validated the inhibition function of sIFNα for the proliferation of A549 cell, showing that the cell activity after treatment by sIFNα is significantly inhibitted, namely, sIFNα could strongly inhibit the proliferation of lung cancer cell A549 (FIG. 2).

### Example 3

### A549 Cell Morphological Observation

A549 cells are paved on 6-pore plate, and treated separately by sIFNα or IFNα2b on the next day so that the final concentration is 7 μg/ml, the control group isn't treated. After 3 d, the cells are put under an inverted microscope (OLYMPUS, IX71, Tokyo, Japan) to observe carefully the morphological change and take pictures.

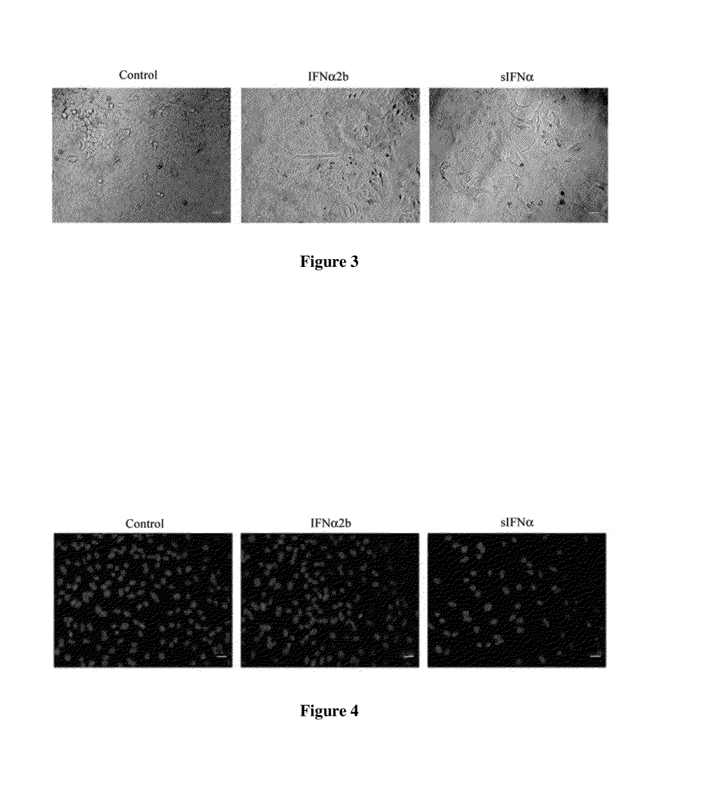

The morphological change of A549 cell after treatment by sIFNα is observed. Referring to FIG. 3, as compared with the control group, the volume of cell in sIFNα treatment group is enlarged obviously, and the cell is distributed on the cell plate in flat and irregular state.

### Example 4

### Hoechst 33258 Staining

A549 cells are inoculated into 24-pore plate with cover glass, and treated separately by 7 μg/ml sIFNα or IFNα2b on the next day, the control group isn't treated. then cultured continuously for 3 d. The culture fluid is removed, then it's flushed with PBS 2 times, and fixed 10 min by 4% PFA, then Hoechst 33258 staining fluid is added, and then washed off after 5 min; the glass is taken out, and the sealing agent for preventing fluorescence quenching is covered on the glass slide; next placed under fluorescence microscope for observation and taking pictures.

The shape of cell nucleus is tested by Hoechst33258 staining. As compared with the control group, it's found that the number of cells in sIFNα treatment group is much smaller, showing stronger anti-proliferative capability; however, no morphological change of the cell nucleus occurs (FIG. 4).

### Example 5

### Western Blot

A549 cells are paved on 10 cm culture plate, and treated separately by 0.1 μg/ml, 1 μg/ml, 7 μg/ml sIFNα or IFNα2b on the next day (other than the treatment group), then put into the incubator for continuous culturing. Cells are collected after 3 d, then cracked by the lysis solution of Beyotime; next, the concentration of protein is measured by BCA method, and balanced to the same level by the lysis solution; then, 6× loading buffer is added, and boiled 10 min in hot water, saved at −80° C. or used immediately for electrophoresis. During sampling, 40 μg protein is added into every pore, and subject to 80V in standard electrophoresis buffer until the stain reaches the bottom of the glue; then, 150 mA flows constantly for 150 min to transfer the protein to PVDF film. The relevant protein expression is measured by relevant antibody.

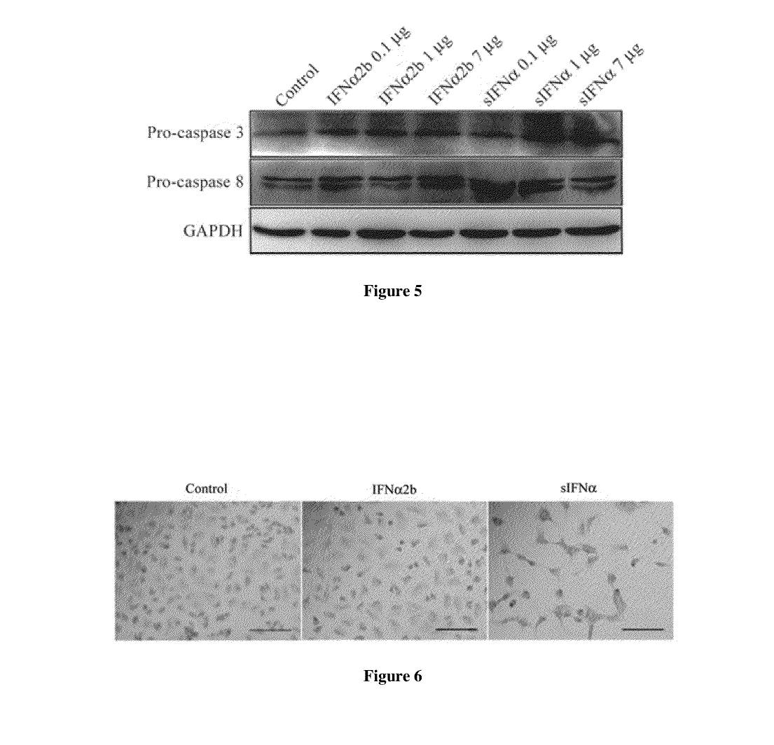

The expressions of apoptosis-related caspase 3 and caspase 8 are tested by the method of Western blot. The results show that, sIFNα treatment group for A549 cell has no effect on the expressions of these two proteins (FIG. 5).

### Example 6

### Tunel Staining

The cells are inoculated into 24-pore plate with cover glass, and treated separately by 7 μg/ml sIFNα or IFNα2b on the next day, the control group isn't treated. After 3 d, cover glass with growing cell is taken out, fixed for 10 min at 4% PFA room temperature; next, the following steps are performed by the instruction of TACS TdT Kit in situ Apoptosis Detection Kit: PBS is flushed 10 min; 50 μl Cytonin™ (Cat# 4876-05-01) is dripped onto every glass sheet for incubation 30 min at room temperature; flushed twice by sterile water, 2 min every time; immersed in 3% hydrogen peroxide methanol solution for 5 min incubation at room temperature; flushed by PBS; 1× TdT Labeling Buffer is dripped for 5 min incubation at room temperature; Labeling Reaction Mix is dripped, then it's placed into 37° C. incubator for 60 min incubation; immersed into 1× TdT Stop Buffer for 5 min incubation at room temperature; flushed by PBS; 50 μl Strep-HRP is dripped onto every glass sheet and placed into 37° C. incubator for 10 min incubation; flushed by PBS; colored 5 min by DAB; then stained by methyl green, dehydrated by gradient ethanol and transparentize d by xylene, and sealed by neutral resin; it's dried up and then placed under upright microscope (OLYMPUS, BX51, Tokyo, Japan) for observation.

The apoptosis is tested by Tunel staining method. The results show that, the cells are Tunel-negative (FIG. 6). At this point, it's confirmed that sIFNα will not cause apoptosis of A549 cell in conjunction with FIGS. 5 and 6.

### Example 7

### SA-β-Gal Staining

Senescence of A549 is tested by cellular senescence β-Galactosidase Staining Kit. Firstly, the cell is paved onto 6-pore plate, with the cell density of 10000/pore; then treated separately by 7 μg/ml sIFNα or IFNα2b on the next day, and the treatment group isn't treated. After 3 d, the cell culture fluid is removed, and washed 1 time by PBS, then 1 ml β-galactosidase Staining Kit is added, and fixed 15 min at room temperature; next, the cell fixative is removed, and washed 3 times by PBS, 3 min every time. PBS is removed, 1 ml staining fluid is added into every pore, incubated overnight at 37° C., then placed under microscope for observation and taking pictures.

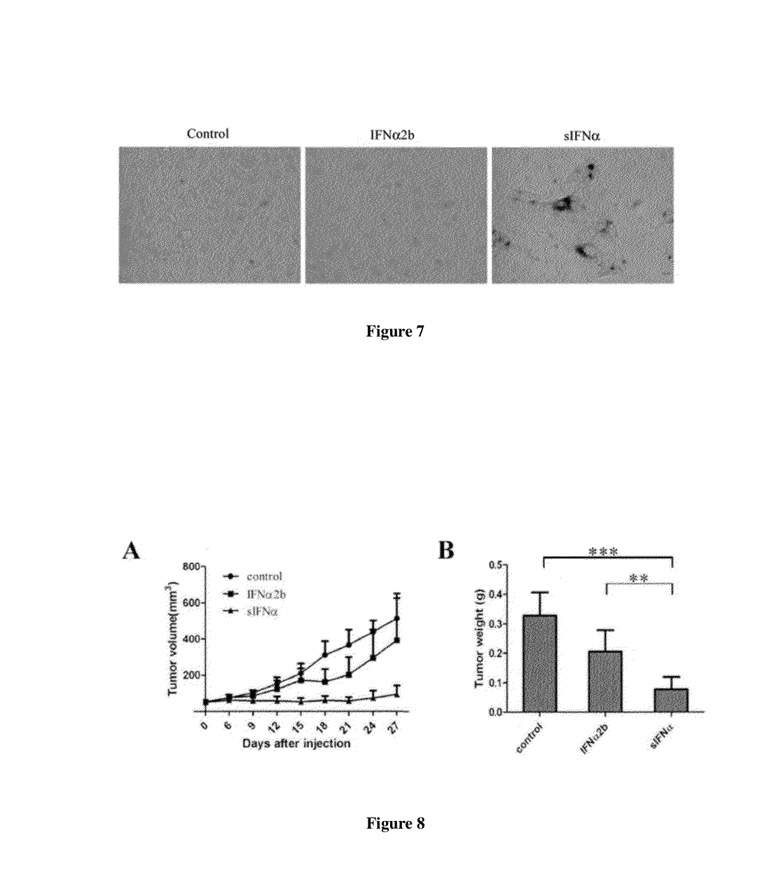

The statistical results show that, about 80% of cells in sIFNα treatment group are SA-β-Gal-positive, and only 20% in IFNα2b treatment group; meanwhile less than 1% cells in the control group are stained (FIG. 7, table 1). That's to say, sIFNα could inhibit the growth of cancer by inducing senescence of lung cancer cell A549.

TABLE 1
The percentages of SA-β-gal positive cells

| Group | SA-β-gal positive (%) | P value |
|---|---|---|
| Control | 0.715 ± 0.721 | P < 0.0001 |
| IFNα2b | 15.526 ± 4.145 | P < 0.0001 |
| sIFNα | 75.458 ± 5.363 | — |

Note:
values are means ± SD of three independent experiments; the statistic significances of the differences between sIFNα and IFNα2b or PBS were shown by P values.

### Example 8

### Inhibiting Growth of Transplanted Tumor in Nude Mice by sIFNα

(1) Primary screening of cell strain in nude mice: the primary screening of tumor cells is helpful to increase the proportion of tumor formation and improve the tumor consistency. Firstly, 4 4-week BALB/c nude mice are ordered, and 10⁶ A549 cells are injected into each one; after about 2 weeks, the nude mice are killed by cervical dislocation, then the tumor is stripped on sterile bench, cut into pieces by sterile ophthalmic scissors, and placed into 10% FBS RPMI-1640 culture medium for primary culturing and extensive proliferation until sufficient experimental tumor cells are acquuired.

(2) Animal test: 40 4-week nude mice are ordered, and 10⁶ A549 tumor cells for primary culturing are injected subcutaneously at right back; they're classified randomly into 3 groups (8 for every group) when the tumor grows to about 100 mm³, then PBS or sIFNα (100 μg/per mouse) or IFNα2b (100 μg/per mouse) is injected into the tumor 12 times on every other day. Observe the growth of tumor and measure the size and volume of tumor by vernier caliper (mm³) (calculation method: length×width×height/2). After the test, strip off the tumor of the nude mice, measure and record separately. The tumor is stored in a freezing tube with liquid nitrogen, and then kept at −80° C.

The analysis of the volume of transplanted tumor shows that, sIFNα can strongly inhibit the transplanted tumor; the transplanted tumor of control group grows quickly to about 500 mm³ within four weeks. However, the transplanted tumor of sIFNα treatment group is always maintained within 100 mm³, with the inhibition rate up to 80% (FIG. 8A). After the test, the nude mice are killed by cervical dislocation, then the transplanted tumor is stripped and measured. The results also show that, the transplanted tumor of sIFNα treatment group is much lighter than that of the control group (FIG. 8B).

### Example 9

### Immune Cell Staining

A549 cell immune staining is tested by Rabbit or Mouse ABC Staining System Kit as per the instructions of the manufacturers: the tumor stripped off from the nude mice in Example 8 is prepared into paraffin sections, which are dewaxed and rehydrated; then, they're treated 10 min by the Hydrogen Peroxide Block in the Kit to quench endogenous peroxidase; flushed twice by PBS, 5 min every time; the sections are placed into PH 6.0 0.01 M citrate buffer, and boiled 10 min in a microwave oven for antigen recovery; next sealed 5 min at room temperature by Protein Block in the Kit; flushed twice by PBS, 5 min every time; an antibody is added, and then they're placed overnight in a wet box at 4° C.; flushed four times by PBS, 5 min every time; Anti-Mouse&Rabbit is dripped for 10 min incubation at room temperature; flushed four times by PBS, 5 min every time; Streptavidin Peroxidase is added for 10 min incubation at room temperature; flushed four times by PBS, 5 min every time; DAB is dripped for 10 min incubation at room temperature; flushed four times by PBS, 5 min every time; restained 2 min by hematoxylin, then dehydrated by gradient ethanol and transparentized by xylene, next sealed by neutral resin; after drying up at room temperature, they're placed under OLYMPUS BX51 microscope for observation and taking pictures.

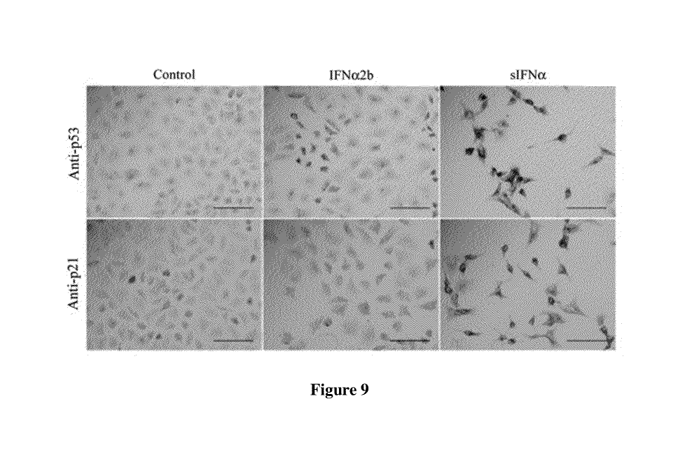

The expression of senescence-related cells is tested by immune cell staining. It's found that, in the cells of sIFNα treatment group, both p53 and p21 rise (FIG. 9), further proving the senescence of A549 cell under the inducement of sIFNα.

What is claimed is:

1. A method to induce cell senescence by a recombinant interferon, comprising the step of contacting target cells with the recombinant interferon, wherein the interferon is encoded by a nucleotide sequence having the sequence of SEQ ID NO.2.

2. The method of claim 1, wherein the cells are cancer cells.

3. The method of claim 1, wherein the interferon does not induce cell apoptosis.

4. The method of claim 1, wherein the interferon exhibits anti-proliferation effect in lung cancer cell A549.

5. The method of claim 1, further comprises the step of examining the cells by crystal violet staining.

6. The method of claim 1, further comprises the step of examining the cells by Hoeschst 33258 staining.

7. The method of claim 6, wherein the cells possess enlarged cell size and flatted shape in morphology.

8. The method of claim 6, wherein the cells do not exhibit any nucleus morphologic changes.

9. The method of claim 6, wherein the number of cells are reduced.

10. The method of claim 1, further comprises the step of examining the cells for caspase 3 or caspase 8 expression.

11. The method of claim 10, wherein the cells do not exhibit any change in caspase 3 or caspase 8 expressions.

12. The method of claim 1, further comprises the step of examining the cells by p53 or p21 immunostaining.

13. The method of claim 12, wherein the cells exhibit increased expression of p53 or p21.

14. The method of claim 1, further comprises the step of examining the cells by Tunel staining.

15. The method of claim 1, further comprises the step of examining the cells by SA-β-gal staining assay.

16. The method of claim 15, wherein the cells become SA-β-gal positive.
